(19) United States
(12) Patent Application Publication
(10) Pub. No.: US 2005/0062724 A1
(43) Pub. Date: Mar. 24, 2005

(54) Display device and method thereof

(76) Inventors: Young-chan Kim, Yelwang-si (KR); Dong-hoon Lee, Seoul (KR)

Correspondence Address: STANZIONE & KIM, LLP, 1740 N STREET, N.W., FIRST FLOOR, WASHINGTON, DC, 20036, US

(21) Appl. No.: 10/927,112

(22) Filed: Aug. 27, 2004

(30) Foreign Application Priority Data
Sep. 24, 2003 (KR) 2003-66390

(51) Int. Cl. G09G 5/00; A61B 5/02

(52) U.S. Cl. 345/168

(57) ABSTRACT

A display device having a display to display a picture thereon, a power supply to supply power to the display, and a parameter setter to set a predetermined adjustment parameter. The display further includes one shared key and a controller. The controller can receive a push input signal from the sheared key, divide a duration time of the push input signal into a plurality of duration time sections, and control the power supply and the parameter setter to turn on/off the display device when the push input signal belongs to a first duration time section, to implement a parameter setting mode when the push input signal belongs to a second duration time section, and to implement a parameter executing mode when the push input signal belongs to a third duration time section. With this configuration, a display device in which only one key operation is enough to control a power supplying system and various adjustment parameters can be set as desired is provided.

### CROSS-REFERENCE TO RELATED APPLICATIONS

This application claims the benefit of Korean Patent Application No. 2003-66390, filed Sep. 24, 2003, in the Korean Intellectual Property Office, the disclosure of which is incorporated herein by reference.

### BACKGROUND OF THE INVENTION

1. Field of the Invention

The present general inventive concept relates to a display device including a display to display a picture thereon, a power supply to supply power to the display, and a parameter setter to set a predetermined adjustment parameter, and a method thereof, and more particularly, to a display device in which only one key operation is enough to control a power supplying system and to set various adjustment parameters as desired, and a method thereof.

2. Description of the Related Art

A conventional display device comprises a display to display a picture thereon, a plurality of on screen display (OSD) buttons allowing a user to adjust a displaying state of the picture, a power button allowing a user to select power to be supplied to the display device, a signal processor receiving a video signal and processing the video signal to be properly displayed as a picture on the display, and a light emitting diode (LED) flashing and allowing a user to know whether the display displays a picture.

Further, the conventional display device comprises an OSD menu storage storing an OSD menu to be displayed when the OSD button is pushed, an OSD state setter to set the displaying state selected by a user through the OSD menu, and other operation buttons and a controller to control the displaying state related to a received video signal and an OSD to be displayed on the display as desired.

In the conventional display device, the display state of a picture can be adjusted by a user through the plurality of OSD buttons placed in a front of the display device.

Meanwhile, some settable adjustment parameters of the display device are set by combining a plurality of key operations, or are allowed to be set by a specific method which can be performed by not a general user, but a professional user such as a manufacturer, a warranty repairer or the like.

To easily set some adjustment parameters whose setting operations are complicated or restricted, if a separate button is additionally provided to set such adjustment parameters, the exterior of the display device should be redesigned regarding the separate button, so that there arise problems that a production line is changed due to a new design, and a production cost is increased.

Furthermore, if the number of OSD buttons provided to adjust the displaying state is minimized, a production cost can be decreased.

### SUMMARY OF THE INVENTION

Accordingly, it is an aspect of the present general inventive concept to provide a display device with a minimum number of operation buttons provided in the display device.

Another aspect of the present general inventive concept is to provide a display device in which only one key operation is enough to control a power supplying system and to set various adjustment parameters as desired.

Additional aspects and/or advantages of the general inventive concept will be set forth in part in the description which follows and, in part, will be obvious from the description, or may be learned by practice of the invention.

The foregoing and/or other aspects of the present general inventive concept are achieved by providing a display device including a display to display a picture thereon, a power supply to supply power to the display, a parameter setter to set a predetermined adjustment parameter, one shared key, and a controller to receive a push input signal from the shared key, to divide a duration time of the push input signal into a plurality of duration time sections, and to control the power supply and the parameter setter to turn on/off the display device when the push input signal belongs to a first duration time section, to implement a parameter setting mode when the push input signal belongs to a second duration time section, and to implement a parameter executing mode when the push input signal belongs to a third duration time section.

According to an aspect of the general inventive concept, the display device further includes an OSD generator allowing an OSD menu having a plurality of parameter setting items to be displayed on the display, wherein the controller controls the OSD generator and the parameter setter to select in sequence the parameter setting items of the OSD menu, or to set an adjustment parameter of the selected parameter setting item according to a duration time of the push input signal when the parameter setting mode is implemented.

According to another aspect of the general inventive concept, the parameter executing mode can be implemented to execute settings of the adjustment parameter different from each other corresponding to of the respective duration time sections which are divided depending on the duration times of the push input signal.

According to another aspect of the general inventive concept, the display device further includes a section change indicator to receive a section change signal from the controller and to indicate a change of the duration time, the section change indicator including an LED or a beeper.

According to still another aspect of the general inventive concept, the duration time is increased in sequence of the first duration time section to turn on/off the display device, the third duration time section to implement the parameter executing mode, and the second duration time section to implement the parameter setting mode.

A display device having a display to display a picture thereon, a power supply to supply power to the display, and a parameter setter to set a predetermined adjustment parameter, the display device including one shared key to generate a push input signal when pushed, and a controller to receive the push input signal from the shared key and to set controls of the display corresponding to durations of time in which the shared key is pushed.

A method of displaying a picture on a display having a power supply to supply power to the display, and a parameter setter to set a predetermined adjustment parameter, the method including dividing a duration of time in which a shared key is pushed into a plurality of duration time sections, and setting controls of the display corresponding to the respective duration time sections.

### BRIEF DESCRIPTION OF THE DRAWINGS

These and other aspects and advantages of the present general inventive concept will become apparent and more readily appreciated from the following description of the embodiments, taken in conjunction with the accompany drawings of which:

FIG. 1 is a perspective view of a display device according to an embodiment of the present general inventive concept;

FIG. 2 is a control block diagram of the display device of FIG. 1;

FIG. 3 is a control flowchart illustrating a method of implementing the display device of FIG. 1, according to another embodiment of the present general inventive concept;

FIG. 4 is a control flowchart of a parameter setting mode in FIG. 3; and

FIG. 5 illustrates an example of an OSD menu displayed on the display of FIG. 1.

### DETAILED DESCRIPTION OF THE PREFERRED EMBODIMENTS

Reference will now be made in detail to the embodiments of the present general inventive concept, examples of which are illustrated in the accompanying drawings, wherein like reference numerals refer to like elements throughout. The embodiments are described below in order to explain the general inventive concept by referring to the figures.

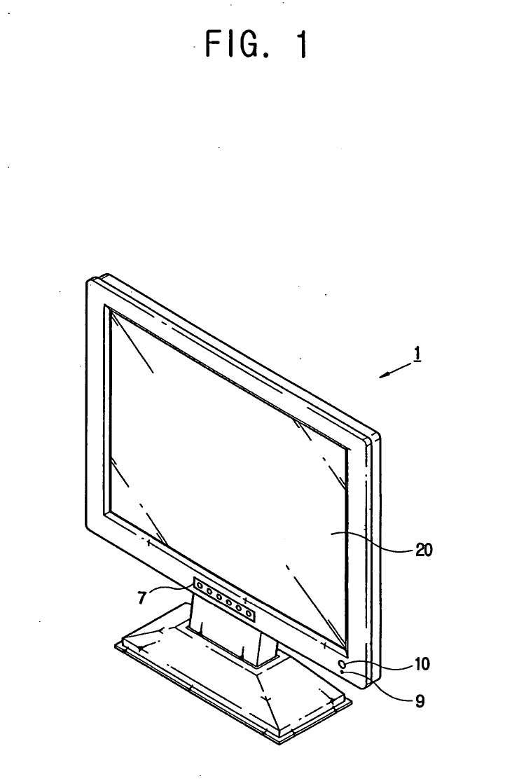

FIG. 1 is a perspective view of a display device according to an embodiment of the present general inventive concept. As shown therein, a display device 1 can include components such as a display 20 to display a picture thereon, a plurality of OSD buttons 7 allowing a user to adjust a displaying state of the picture, etc., which overlap with the conventional display device, and a shared key 10 allowing a user to select power to be supplied to the display device 1 or to selectively set or execute a plurality of adjustment parameters.

Further, like the conventional display device, the display device according FIG. 1 can include a signal processor (not shown) to receive a video signal and processing the video signal to be properly displayed as a picture on the display 20, and an LED 9 to flash and allow a user to know whether the display 20 is displaying a picture.

In addition, the display device according to this embodiment can include an OSD menu storage (not shown) to store an OSD menu to be displayed when the OSD button 7 is pushed, an OSD state setter (not shown) to set the displaying state selected by a user through the OSD menu, and other operation buttons (not shown) and a controller to control the displaying state related to a received video signal and an OSD to be displayed on the display as desired.

The shared key 10 can be employed as a key that allows a user to select power to be supplied to the display device 1 or to selectively set or execute a plurality of adjustment parameters, which transmits a push input signal, generated when a user pushes the shared key 10, to the controller (to be describe later), thereby allowing the controller to determine which function to be operated depending on a duration time of the push input signal.

Here, the duration time of the push input signal is defined as the time from when the shared key 10 is pushed to when the shared key 10 is released from being pushed. That is, the duration time can be calculated on the basis of a change of the push input signal from a high state (or a low state) when the shared key 10 is pushed to the low state (or the high state) when the shared key 10 is released from being pushed.

In FIG. 1, the shared key 10 can be placed in a front right lower part of the display device 1, but is not limited thereto, and may be attached to any location of the display device 1, or can be connected to the display device 1 via an external communication assisting member, such as, for example, a cable.

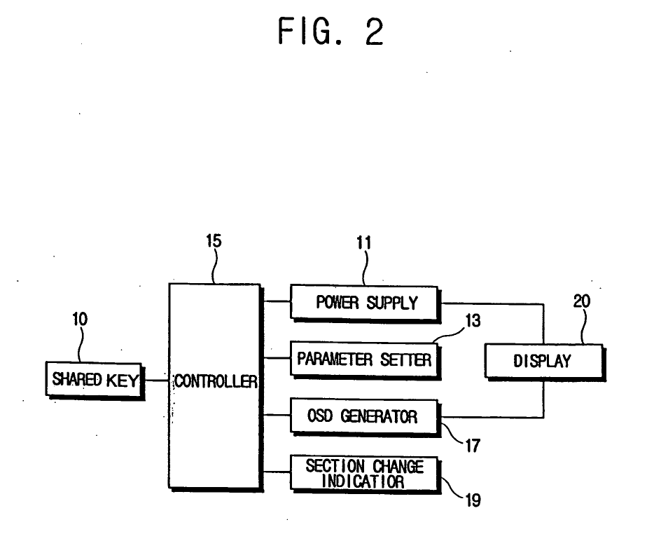

FIG. 2 is a control block diagram of the display device of FIG. 1. As shown in FIG. 2, the display device 1 can include a display 20 to display a picture thereon, a power supply 11 to supply power to the display 20, a parameter setter 13 to set a predetermined adjustment parameter, one shared key 10, and a controller 15 to receive the push input signal from the shared key 10, to divide the duration time of the push input signal into a plurality of duration time sections, and to control the power supply 11 and the parameter setter 13 to turn on/off the display device 1 when the push input signal belongs to a first duration time section, to implement a parameter setting mode when the push input signal belongs to a second duration time section, and to implement the parameter executing mode when the push input signal belongs to a third duration time section.

Further, the display device 1 can include an OSD generator 17 to allow the OSD menu, having a plurality of items (or parameter setting items), to respectively set the adjustment parameters to be displayed on the display 20, and a section change indicator 19 to indicate a change of the duration time section to which the push input signal belongs.

The parameter setter 13 can be employed to set predetermined adjustment parameters. Thus, adjustment parameters may be set which are not easy for a general user to set, such as some adjustment parameters of the conventional display device.

In an aspect of the general inventive concept, the adjustment parameters can relate to settings such as an auto-source selection to automatically set the video signal as an analog signal or a digital signal after determining whether the received video signal is the analog signal or the digital signal, respectively, an auto-screen adjustment to automatically adjust a screen, a beeper setting to set whether a beeper sounds or not when the shared key 10 is pushed, and an LED setting to set whether the LED 9 (refer to FIG. 1), which indicates whether a picture is displayed on the display 20, flashes or is turned off when a picture is not displayed on the display 20.

In this embodiment, it is an aspect that the adjustment parameters can include an adjustment parameter related to a settable attribution for the shared key 10 newly added according to an embodiment of the present general inventive concept as well as the adjustment parameters which are not easy for a general user to set, such as some adjustment parameters of the conventional display device. However, the adjustment parameters are not limited thereto, and may include any adjustment parameter having the settable attribution.

Further, the adjustment parameters may include the adjustment parameter related to the displaying state which can be set by the OSD button of the conventional display device. In this case, the plurality of OSD buttons 7 are not provided in a front portion of the display device 1.

The controller 15 can receive the push input signal from the shared key 10. Further, the controller 15 can control the power supply 11 and the parameter setter 13 to turn on/off the display device 1 depending on the duration time of the received push input signal, to implement the parameter setting mode to set the adjustment parameter, or to implement a parameter executing mode to execute the adjustment parameter as it is set.

To achieve the foregoing controls, the duration time of the push input signal can be divided into a plurality of duration time sections, and the controls can be implemented differently according to the duration time sections, respectively. Here, the controls include turning on/off the display device 1, implementing the respective parameter executing modes to execute the adjustment parameters different from each other, and to implement the respective parameter setting modes to set the adjustment parameters different from each other.

The controls according to the duration time sections are as follows.

TABLE 1

| | Duration time (sec.) of | |
|---|---|---|
| Section | the push input signal | Control |
| First section | 0 < t < 1 | System power on/off |
| Second section | 1 ≦ t < 2 | Auto-source selection |
| Third section | 2 ≦ t < 3 | Auto-screen adjustment |
| Fourth section | 3 ≦ t < 4 | Parameter setting mode |

As shown in Table 1, in the case where the duration time of the push input signal is less than 1 second, the display device 1 can be turned on and off from the off and on states, respectively; in the case of the duration time being from 1 second to 2 seconds, the auto-source selection can be implemented to automatically set the video signal as the analog signal or the digital signal after determining whether the received video signal is the analog signal or the digital signal, respectively; in the case of the duration time being from 2 seconds to 3 seconds, the auto-screen adjustment can be implemented to automatically adjust a focus of the screen; and in the case of the duration time being from 3 seconds to 4 seconds, the parameter setting mode can be implemented.

In<Table 1>, the duration time of the push input signal, the number of the duration time sections, and the controls according to the duration time sections are shown by way of example only, and are not limited thereto, and may vary as necessary.

Further, in the <Table 1>, the auto-source selection and the auto-screen adjustment can belong to the parameter executing mode, so that they are set or implemented through the OSD menu in the parameter setting mode.

Meanwhile, when the parameter setting mode is implemented, the OSD menu having the parameter setting items can be displayed on the display 20 so as to allow a user to set the adjustment parameter, wherein the OSD menu is generated by the OSD generator 17.

The OSD generator 17 can allow movement in sequence among the parameter setting items of the OSD menu, or can illustrate the parameter setting items related to the settable adjustment parameters which can be set by a user, according to the duration time of the push input signal, thereby allowing a user to select the parameter setting items and to change the settings of the adjustment parameters corresponding to the selected parameter setting items.

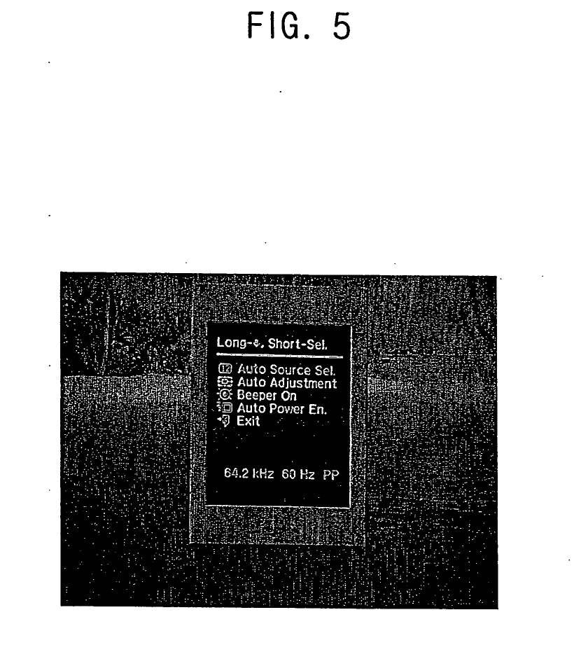

An example of the OSD menu displayed on the display 20 is illustrated in FIG. 5, and the adjustment parameter corresponding to each parameter setting item and the settings of the adjustment parameter about the selected parameter setting item will be described with reference to the following <Table 2>.

TABLE 2

| | Parameter | |
|---|---|---|
| | setting item | |
| Item | (refer to FIG. 5) | Adjustment parameter |
| First item | Auto Source Sel. | Auto-source selection |
| Second item | Auto Adjustment | Auto-screen adjustment |
| Third item | Beeper On | Beeper on/off |
| Fourth item | Auto Power En. | LED on/off |
| Fifth item | Exit | OSD menu quit |

As shown in<Table 2>, the first item relates to the adjustment parameter for the auto-source selection to automatically set the video signal as the analog signal or the digital signal after determining whether the received video signal is the analog signal or the digital signal, respectively; the second item relates to the adjustment parameter for the auto-screen adjustment to automatically adjust the screen; the third item relates to the adjustment parameter for the beeper setting to set whether a beeper is operated or not when the shared key 10 is pushed; the fourth item relates to the adjustment parameter for the LED setting to set whether the LED 9 (refer to FIG. 1) flashes, which indicates whether a picture is displayed on the display 20, or is turned off, which indicates when a picture is not displayed on the display 20; and the fifth item relates to the control allowing a user to exit from the OSD menu.

The number of items and the kind of adjustment parameters in<Table 2> are shown by way of example only, and are not limited thereto and may vary as necessary.

In the case of a parameter setting mode in which the OSD menu of FIG. 5 is displayed, the item selection or the item setting are implemented according to the duration time of a user's push input signal. For example, the item selection can be implemented when the duration time is relatively long, and the item setting can be implemented when the duration time is relatively short. The long duration time (i.e., the shared key 10 is pushed for a long time) and the short duration time (i.e., the shared key 10 is pushed for a short time) are distinguished with respect to a predetermined section change reference time.

To allow a user to know the duration time section, the section change indicator 19 can operate the LED or the beeper whenever the duration time section is changed.

That is, the controller 15 can receive the push input signal and can count the duration time elapsed since the shared key 10 has been pushed. While the controller 15 counts the duration time, the controller 15 can transmit a section change signal to the section change indicator 19 whenever the counted duration time reaches the predetermined section change reference time. Thus, the section change indicator 19 can receive the section change signal from the controller 15 and can indicate the change of the duration time.

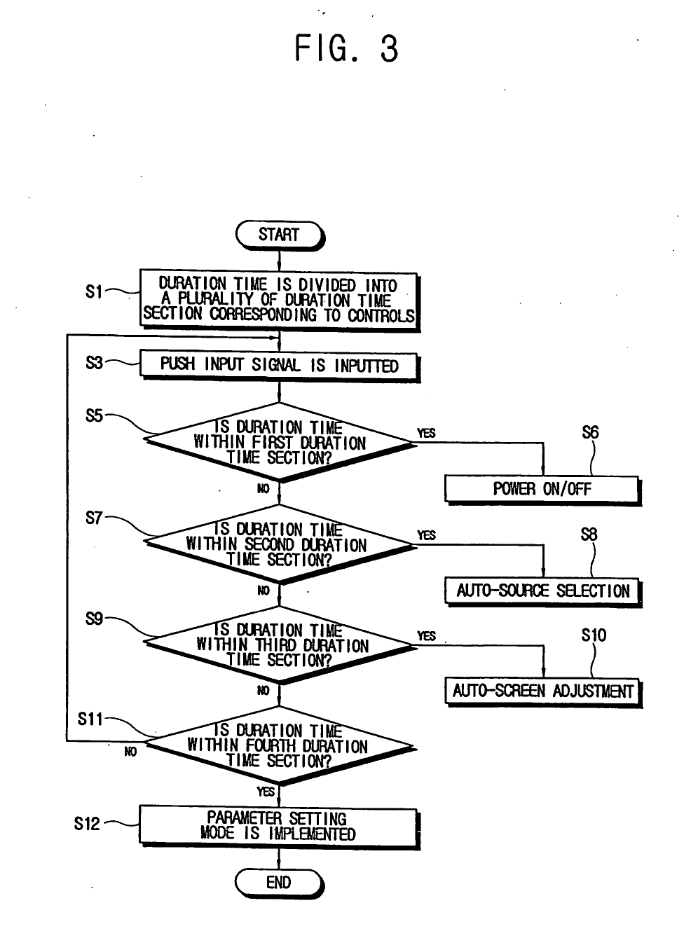

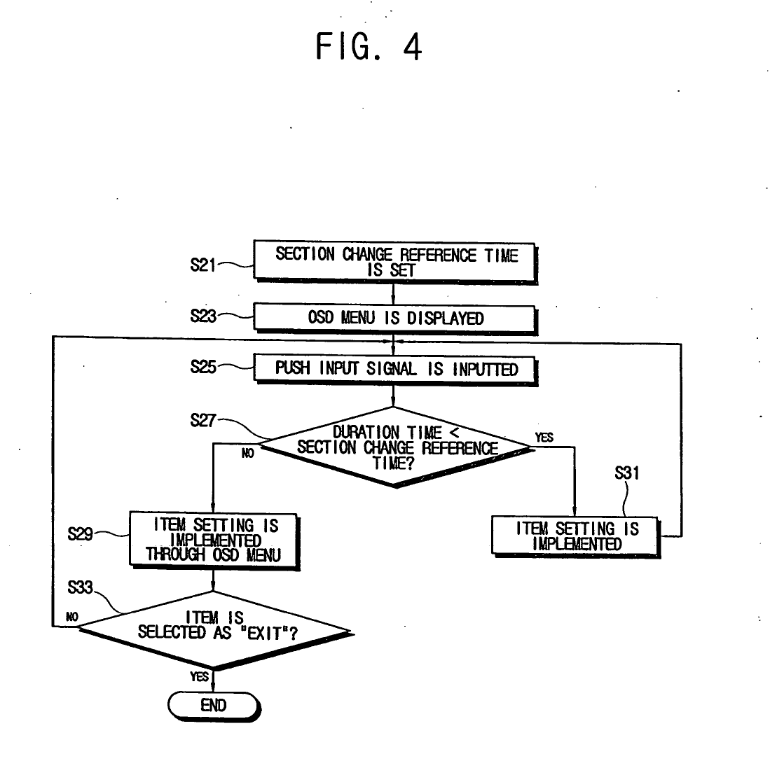

FIGS. 3 and 4 are control flowcharts of the display device according to an embodiment of the present invention. This embodiment is based on<Table 1>, <Table 2> and FIG. 5. As shown in FIG. 3, at operation S1, the duration time of the push input signal is divided into the plurality of duration time sections, and the controls are set corresponding to the respective duration time sections.

At operation S3, the shared key 10 is pushed by a user. The duration time of the push input signal is counted until the push input signal is changed as the user releases the shared key 10.

At operation S5, it is determined whether the duration time is within the first duration time section. When the duration time is within the first duration time section, at operation S6, the control corresponding to the first duration time section is implemented, that is, the power supply is controlled to turn on/off the display device 1.

When the duration time is not within the first duration time section, at operation S7, it is determined whether the duration time is within the second duration time section. When the duration time is within the second duration time section, at operation S8, the control corresponding to the second duration time section is implemented, that is, the auto-source selection is implemented.

When the duration time is not within the second duration time section, at operation S9, it is determined whether the duration time is within the third duration time section. When the duration time is within the third duration time section, at operation S10, the control corresponding to the third duration time section is implemented, that is, the auto-screen adjustment is implemented.

When the duration time is not within the third duration time section, at operation S11, it is determined whether the duration time is within the fourth duration time section. When the duration time is within the fourth duration time section, at operation S12, the control corresponding to the fourth duration time section is implemented, that is, the parameter setting mode is implemented.

As shown in FIG. 4, the parameter setting mode S12 of FIG. 3 can be implemented as follows. Here, at operation S21, the section change reference time should be set forth beforehand. For example, the section change reference time is preferably set at the same time when the operation S1 is performed.

The implementation of the parameter setting mode can be started with displaying the OSD menu having the plurality of parameter setting items on the display 20, at operation S23.

In the state that the OSD menu is displayed as shown in FIG. 5, at operation S25, a user pushes the shared key 10 for a long or short duration time. Here, the long duration time and the short duration time are distinguished with respect to a predetermined section change reference time.

At operation S27, the duration time elapsed since the shared key 10 is pushed is compared with the predetermined section change reference time. When the duration time is shorter than the section change reference time, at operation S29, the item selection among the OSD menu is implemented. When the duration time is longer than the section change reference time, at operation S31, the item setting corresponding to the selected item is implemented, for example, “on” of the adjustment parameter corresponding to the selected item is changed to “off”, and vice versa. At this time, the setting change can be applied by the parameter setter 13 at the same time the adjustment parameter is set, or can be applied when a user exits the OSD menu.

That is, both the operations S29 and S31 are performed by pushing the shared key 10 once, and then the controller 15 waits until the shared key 10 is pushed again. While performing the operation S29, it is determined whether the parameter setting item is selected as “EXIT” or not, at operation S33. When the parameter setting item is selected as “EXIT”, the OSD menu is ended, thereby completing the whole control.

With this configuration, the parameter setting mode to turn on/off the display device or to set the adjustment parameter, and the parameter executing mode to execute the adjustment parameters are implemented according to the duration times elapsed since one shared key is pushed by a user, so that only one key operation is enough to control a power supplying system and to set various adjustment parameters as desired.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A display device having a display to display a picture thereon, a power supply to supply power to the display, and a parameter setter to set a predetermined adjustment parameter, the display device comprising:
one shared key; and
a controller to receive a push input signal from the shared key, to divide a duration time of the push input signal into a plurality of duration time sections, and to control the power supply and the parameter setter to turn on/off the display device when the push input signal belongs to a first duration time section, to implement a parameter setting mode when the push input signal belongs to a second duration time section, and to implement a parameter executing mode when the push input signal belongs to a third duration time section.

2. The display device according to claim 1, further comprising an OSD generator to allow an OSD menu having a plurality of parameter setting items to be displayed on the display,
wherein the controller controls the OSD generator and the parameter setter to select in sequence the parameter setting items of the OSD menu, or to set an adjustment parameter of the selected parameter setting item according to the duration time of the push input signal when the parameter setting mode is implemented.

3. The display device according to claim 1, wherein the parameter executing mode is implemented to execute settings of the adjustment parameter different from each other corresponding to the plurality of duration time sections which are divided depending on the duration times of the push input signal.

4. The display device according to claim 3, further comprising a section change indicator to receive a section change signal from the controller and to indicate a change of the duration time, the section change indicator comprising an LED or a beeper.

5. The display device according to claim 1, wherein the duration time is increased in sequence of the first duration time section to turn on/off the display device, the third duration time section to implement the parameter executing mode, and the second duration time section to implement the parameter setting mode.

6. The display device according to claim 1, wherein the adjustment parameters include an auto-source selection to automatically set a received video signal as an analog signal or a digital signal after determining whether the received video signal is the analog signal or the digital signal, an auto-screen adjustment to automatically adjust a screen, a beeper setting to set whether a beeper sounds or not when the shared key is pushed, and an LED setting to set whether the LED flashes or is turned off corresponding to whether a picture is displayed on the display.

7. The display device according to claim 1, wherein when the duration time of the push input signal is less than 1 second, the display device can be turned on and off from the off and on states, respectively; when the duration time of the push input signal is one second to two seconds, the auto-source selection can be implemented to automatically set a video signal as the analog signal or the digital signal after determining whether the received video signal is an analog signal or a digital signal, respectively; when the duration time of the push input signal is two seconds to three seconds, the auto-screen adjustment can be implemented to automatically adjust a focus of the screen; and when the duration time of the push input signal is three seconds to four seconds, the parameter setting mode can be implemented.

8. A display device having a display to display a picture thereon, a power supply to supply power to the display, and a parameter setter to set a predetermined adjustment parameter, the display device comprising:
one shared key to generate a push input signal when pushed; and
a controller to receive the push input signal from the shared key and to set controls of the display corresponding to durations of time in which the shared key is pushed.

9. The display device according to claim 8, wherein the controller controls the power supply to turn on/off the display device when the push input signal belongs to a first duration time section, and controls the parameter setter to implement a parameter setting mode when the push input signal belongs to a second duration time section and to implement a parameter executing mode when the push input signal belongs to a third duration time section.

10. The display device according to claim 9, further comprising an OSD generator to allow an OSD menu having a plurality of parameter setting items to be displayed on the display,
wherein the controller controls the OSD generator and the parameter setter to select in sequence the parameter setting items of the OSD menu, or to set the adjustment parameter of the selected parameter setting item according to the duration time of the push input signal when the parameter setting mode is implemented.

11. The display device according to claim 9, wherein an auto-source selection and an auto-screen adjustment belong to the parameter executing mode and are set or implemented through an OSD menu in the parameter setting mode.

12. A method of displaying a picture on a display having a power supply to supply power to the display, and a parameter setter to set a predetermined adjustment parameter, the method comprising:
dividing a duration of time in which a shared key is pushed into a plurality of duration time sections; and
setting controls of the display corresponding to the respective duration time sections.

13. The method according to claim 12, wherein the controls include an auto-source selection to automatically set a received video signal as an analog signal or a digital signal after determining whether the received video signal is the analog signal or the digital signal, an auto-screen adjustment to automatically adjust a screen, a beeper setting to set whether a beeper sounds or not when the shared key is pushed, and an LED setting to set whether the LED flashes or is turned off corresponding to whether a picture is displayed on the display.

14. The method according to claim 13, wherein when the duration time of the push input signal is less than one second, the display device can be turned on and off from the off and on states, respectively; when the duration time of the push input signal is one second to two seconds, the auto-source selection can be implemented to automatically set the video signal as the analog signal or the digital signal after determining whether the received video signal is the analog signal or the digital signal, respectively; when the duration time of the push input signal is two seconds to three seconds, the auto-screen adjustment can be implemented to automatically adjust a focus of the screen; and when the duration time of the push input signal is three seconds to four seconds, the parameter setting mode can be implemented.

15. The method according to claim 12, wherein when the duration time is within a first duration time section, the control corresponding to the first duration time section is implemented, when the duration time is not within the first duration time section, it is determined whether the duration time is within a second duration time section, and when the duration time is within the second duration time section, the control corresponding to the second duration time section is implemented, and when the duration time is not within the second duration time section, it is determined whether the duration time is within a third duration time section, and when the duration time is within the third duration time section, the control corresponding to the third duration time section is implemented, and when the duration time is not within the third duration time section, it is determined whether the duration time is within a fourth duration time section, and when the duration time is within the fourth duration time section, the control corresponding to the fourth duration time section is implemented.

16. The method according to claim 15, wherein the control corresponding to the first duration time section is turning on and off the power supply, the control corresponding to the second duration time section is an auto-source selection, the control corresponding to the third duration time section is an auto-screen adjustment, and the control corresponding to the fourth duration time section is the parameter setting mode.

17. A display device having a display to display a picture thereon, comprising:
a shared key to select a power on/off mode and a parameter setting mode; and
a controller to alternatively control the display in one of the power on/off mode and the parameter setting mode according to a duration time of the shared key.

18. The display device according to claim 17, wherein the display comprises an OSD button to adjust a display state of the display, and the power on/off mode and the parameter setting mode do not include a displaying state adjusting mode.

19. The display device according to claim 17, wherein the time duration comprises a first time duration and a second time duration, and the controller controls the display in the power on/off mode according to the first time duration and in the parameter setting mode according to the second time duration.

20. The display device according to claim 19, wherein the first time duration is shorter than the second time duration.
